(19) United States
(12) Patent Application Publication
(10) Pub. No.: US 2005/0049598 A1
(43) Pub. Date: Mar. 3, 2005

(54) Suture pulley for use with graft tensioning device

(76) Inventors: Hugh S. West JR., Salt Lake City, UT (US); John R. West, Cincinnati, OH (US); Mark Dallara, Tampa, FL (US)

Correspondence Address: John M. Guynn;WORKMAN, NYDEGGER & SEELEY, 1000 Eagle Gate Tower, 60 East South Temple, Salt Lake City, UT, 84111, US

(21) Appl. No.: 10/786,215

(22) Filed: Feb. 25, 2004

Related U.S. Application Data
(63) Continuation-in-part of application No. 10/651,671, filed on Aug. 29, 2003.

(51) Int. Cl. A61B 17/56

(52) U.S. Cl. 606/72

(57) ABSTRACT

A suture pulley assembly includes a pulley wheel that is rotatably attached to an adjustable tension applicator used in joint repair surgery (e.g., to repair an ACL) so as to equalize the tensile load that is applied to each side of a looped suture by the adjustable tension applicator. The pulley wheel includes a pair of pulley plates that define a pulley space therebetween into which a looped suture is placed and that are oppositely-biased in order to spread apart and contract in response to differences in size of the looped suture as a result of, e.g., the existence of a knot or half knot. The pulley wheel is rotatably attached to the tension applicator by a post. A spring biases one of the suture pulley plates toward the other in a manner that allows for the aforementioned expansion of the pulley space.

### CROSS-REFERENCE TO RELATED APPLICATIONS

This application is a continuation-in-part of copending U.S. application Ser. No. 10/651,671, filed Aug. 29, 2003. The disclosure of the foregoing application is incorporated herein in its entirety.

### BACKGROUND OF THE INVENTION

1. The Field of the Invention

The present invention is in the field of graft tensioning devices used in joint repair surgery, such as reconstruction of the anterior cruciate ligament (ACL). More particularly, the invention relates to improved suture pulleys for use with graft tensioning devices.

2. The Relevant Technology

Graft tension in ACL reconstruction is recognized as an important factor in the clinical outcome of the ACL reconstruction procedure. Grafts that are too loose may be unstable, and grafts that are too tight may greatly restrict motion of the knee. Publications that have emphasized the need for adequate tensioning of the graft include Markolf et al., “Biomechanical Consequences of Replacement of the Anterior Cruciate Ligament With a Patellar Ligament Allograft. Part Two: Forces in the Graft Compared with Forces in the Intact Ligament,” J. Bone Joint Surg. Am., 78:11, 1728-34 (November 1996); Tohyama et al., “Significance of Graft Tension in Anterior Cruciate Ligament Reconstruction. Basic background and clinical outcome,” Knee Surg. Sports Traumatol. Arthroscopy, 6 Suppl. 1, S30-7 (1998); Andersen et al., “Review on Tension in the Natural and Reconstructed Anterior Cruciate Ligament,” Knee Surg. Sports Traumatol Arthroscopy, 2:4, 192-202 (1994); Yasuda et al., “Effects of Initial Graft Tension on Clinical Outcome After Anterior Cruciate Ligament Reconstruction. Autogenous Doubled Hamstring Tendons Connected in Series of Polyester Tapes,” Am. J. Sports Med., 25:1, 99-106 (January 1997). The foregoing publications are incorporated herein by reference.

Devices used to apply a known load to a soft tissue graft are set forth in U.S. Pat. No. 4,712,542; U.S. Pat. No. 5,037,426; U.S. Pat. No. Re 34,762; U.S. Pat. No. 5,713,897; U.S. Pat. No. 5,507,750; and U.S. Pat. No. 5,562,668. For purposes of disclosing mechanisms for applying a known load or. tension onto a soft tissue graft, the foregoing patents are incorporated herein by reference.

A study by Hamner et al. has added to the understanding of graft tension by demonstrating that unequal tension in the individual strands of the soft tissue graft can result in significant losses in total graft strength and stiffness. Hamner et al., “Hamstring Tendon Grafts for Reconstruction of the Anterior Cruciate Ligament: Biomechanical Evaluation of the Use of Multiple Strands and Tensioning Techniques,” J. Bone Joint Surg. Am., 81:4, 549-57 (April 1999). Hamner et al. found that tensioning the soft tissue strands by hand would result in equalization of the load borne by each strand, and that this method was not effective in equalizing the load on the strands, which led to an ultimate graft strength that was not significantly greater than the strength of the individual strands taken alone.

Apparatus and methods for separately applying a load to and conditioning different strands of a multiple-strand soft tissue graft are disclosed in U.S. application Ser. No. 09/711,488, filed Nov. 13, 2000 in the name of Hugh S. West, Jr. and John R. West and entitled “Apparatus and Methods for Independently Conditioning and Pretensioning a Plurality of Ligament Grafts During Joint Repair Surgery”. For purposes of disclosure, the foregoing application is incorporated by reference. The apparatus and methods disclosed in the foregoing application represent a major breakthrough in joint repair apparatus and techniques.

Notwithstanding the foregoing, there is a continuous need to find improvements to apparatus and methods used during joint repair surgery, particularly in equalizing the load on both sides of a looped suture strand attached to a graft tensioning device.

### SUMMARY OF THE INVENTION

The invention encompasses improved suture pulleys that form part of graft tensioning devices. The graft tensioning devices are, in turn, used in conditioning and pre-tensioning a multiple-strand soft tissue graft during joint repair surgery, such as in procedures used to replace or augment the anterior cruciate ligament (“ACL”).

The improved suture pulleys include oppositely biased (e.g., spring loaded) plates that define a groove therebetween into which a suture loop can be placed. The oppositely biased suture pulleys are designed to engage suture loops in a desired manner. In one aspect, providing a suture pulley that is able to expand to increase the size of the interior slot, and then contract as a result of opposed biasing, provides at least one of the following benefits: (1) the expandable suture plates of the suture pulley assist in tying a suture knot by holding the half-knot in place while the second part of the knot is being tied by clamping onto the half knot once made, thereby obviating the need to physically hold the half knot by other means (e.g., with external suture clamps); and (2) the expandable pulley is able to expand or contract depending on the size of the suture strand and any suture knots in order to avoid catching or binding any suture knots that may enter the pulley groove during equalization of the tensile load on each side of a looped suture, thereby helping to better equalize the load applied to each side of the looped suture, which, in turn, helps to equalize the load applied to two ends of a looped soft tissue graft.

The suture pulleys disclosed herein may be used in combination with any graft tensioning device known in the art. They are particularly well-suited for use with graft tensioning devices designed to separately condition and pre-tension multiple strands of a soft tissue graft. Examples of graft tensioning devices with which the inventive suture pulleys can be used are disclosed in U.S. application Ser. No. 10/651,671, filed Aug. 29, 2003, and U.S. application Ser. No. 09/711,488, filed Nov. 13, 2000, both of which were previously incorporated by reference. The graft tensioning devices disclosed therein include a plurality of independently adjustable tension applicators.

In one embodiment, a spring-loaded pulley is attached to each of a plurality of adjustable tension applicators of a graft tensioning device. This allows each suture loop to engage a corresponding suture pulley attached to a respective adjustable tension applicator. When in use, each suture pulley equalizes the load on each side of the particular looped suture strand attached thereto. The ability of the suture pulley to spread apart so as to increase the size of the pulley groove helps any knots to smoothly and freely rotate around the pulley while the load on each end of the suture loop is equalized.

These and other advantages and features of the present invention will become more fully apparent from the following description and appended claims, or may be learned by the practice of the invention as set forth hereinafter.

### BRIEF DESCRIPTION OF THE DRAWINGS

In order that the manner in which the above-recited and other advantages and objects of the invention are obtained, a more particular description of the invention briefly described above will be rendered by reference to a specific embodiment thereof which is illustrated in the appended drawings. Understanding that these drawings depict only a typical embodiment of the invention and are not therefore to be considered to be limiting of its scope, the invention will be described and explained with additional specificity and detail through the use of the accompanying drawings, in which:

FIG. 1 is a perspective view of a graft tensioning device designed to independently condition and pre-tension a multiple-strand soft tissue graft;

FIG. 2 is a cross-sectional view of the graft tensioning device of FIG. 1 taken along line 2-2;

FIG. 2A shows the tensioning device of FIG. 2 after compression of the biasing spring to increase to a tensile load exerted by the tensioning piston onto a looped suture attached at one end to a suture pulley and at another end to multiple ends of a soft tissue graft;

FIG. 3 shows a graft tensioning device attached to a patient's leg during joint repair surgery, with a multiple-strand soft tissue graft emerging from a bone tunnel and looped sutures engaging respective pulleys of the tensioning device;

FIG. 4 is an exploded view of a suture pulley assembly that can be used in combination with, or as part of, a graft tensioning device;

FIG. 5 is a perspective view of the suture pulley assembly of FIG. 4 in assembled form engaging a looped suture;

FIG. 6 is a cross-sectional view of the suture pulley assembly of FIG. 5 taking along line 6-6;

FIG. 7 shows another embodiment of a graft tensioning device attached to a patient's leg, and a soft tissue graft with attached sutures emerging from the bone tunnel;

FIG. 8 shows a pair of looped sutures looped around respective suture pulleys of a graft tensioning device and being secured with suture clamps;

FIG. 9 shows the act of adjusting the tension applied to each looped suture by rotating an adjustment knob on the graft tensioning device, with the suture pulleys equalizing the load applied to each end of the looped suture; and

FIG. 10 illustrates the act of inserting an interference screw into the bone tunnel in order to secure the multiple-strand soft tissue graft against the bone tunnel wall.

### DETAILED DESCRIPTION OF THE PREFERRED EMBODIMENTS.

The invention relates to an improved suture pulley for use with, or that forms part of, a graft tensioning device. In one embodiment, the improved suture pulley is used with, or forms part of, an apparatus suitable for independently conditioning and pre-tensioning a plurality of soft tissue grafts (e.g., two) during joint repair procedures, such as in procedures to replace or augment the anterior cruciate ligament (ACL).

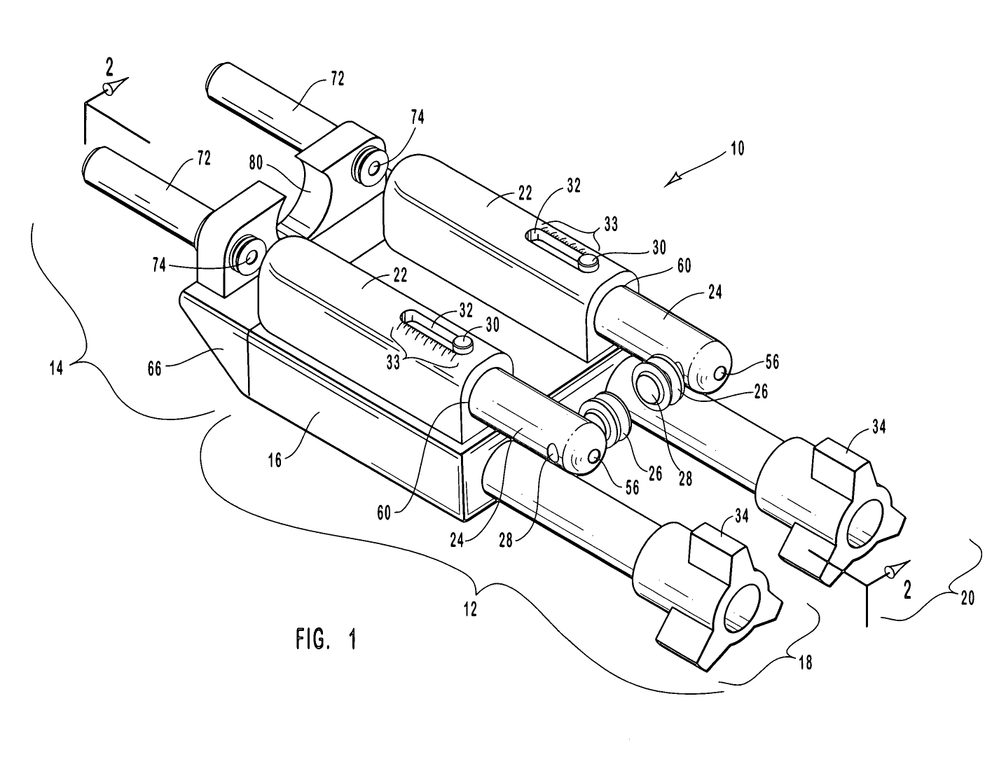

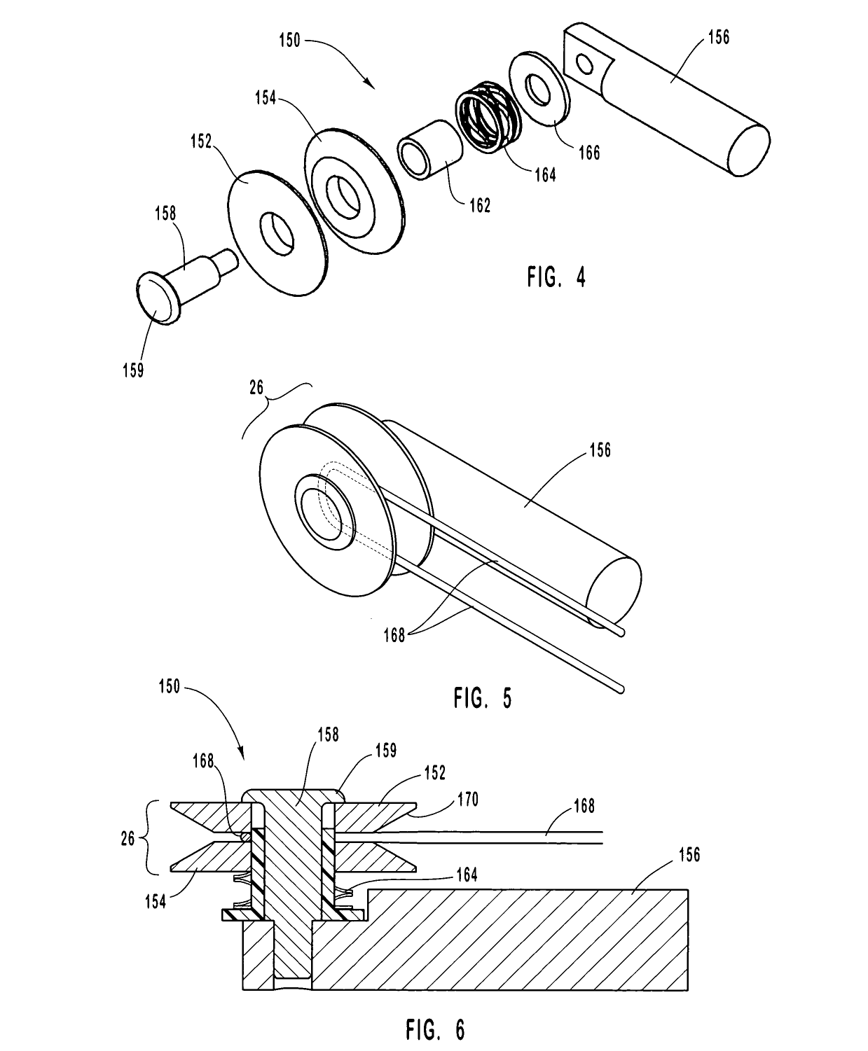

An exemplary suture pulley assembly is illustrated in FIGS. 4-6, which are discussed in detail below. Prior to introducing and discussion FIGS. 4-6, attention is made to FIGS. 1-3, which depict an exemplary graft tensioning device and system with which the inventive suture pulley assembly may be used, or which the inventive suture pulley assembly may form part of.

FIGS. 1-3 depict, by way of background, a graft tensioning device 10 capable of independently conditioning and applying tension to two separate soft tissue strands, or groups of strands. Tensioning device 10 is modular, i.e., it includes two separate and detachable substructures or systems, namely a tensioning system 12 and a limb attachment system 14. Tensioning system 12 includes a tensioning block or module 16. Attached to, or associated with, the tensioning module 16 are a first adjustable tension applicator 18 and a second adjustable tension applicator 20, which are essentially mirror images of each other. Each of the first and second adjustable tension applicators 18 and 20 includes a cylinder block or module 22 and a tensioning piston 24 partially disposed within the cylinder module 22. The cylinder module 22 and tensioning piston 24 are able to move relative to each other.

Each tensioning piston 24 further includes a suture pulley wheel 26 attached by means of a post or axle 28 to the tensioning piston 24. The suture pulley wheel 26 is able to rotate, and thereby self-adjust, after looped sutures have been tied and looped around the suture pulley wheel 26. This ability of the suture pulley wheel 26 to rotate ensures that equal tension is applied to each side of the looped suture. This, in turn, equalizes the tension applied to each end of a looped tissue graft strand. An improved suture pulley assembly 150 comprising said suture pulley wheel is discussed hereafter with respect to FIGS. 4-6.

A tension post 30 attached to the tensioning piston 24 extends through, and freely moves within, a tension indicator slot 32 within the cylinder module 22. The magnitude of the tensile load being applied to a soft tissue graft strand at any given time will be related to the location of the tension post 30 relative to the cylinder module 22. In order to more accurately determine the exact load being applied, graduations 33 may be provided on the cylinder module 22 at or near the tension indicator slot 32. The graduations 33 can provide any desired measuring standard, such as metric (e.g., Newtons) or English units (e.g., pounds), as well as any desired level of precision.

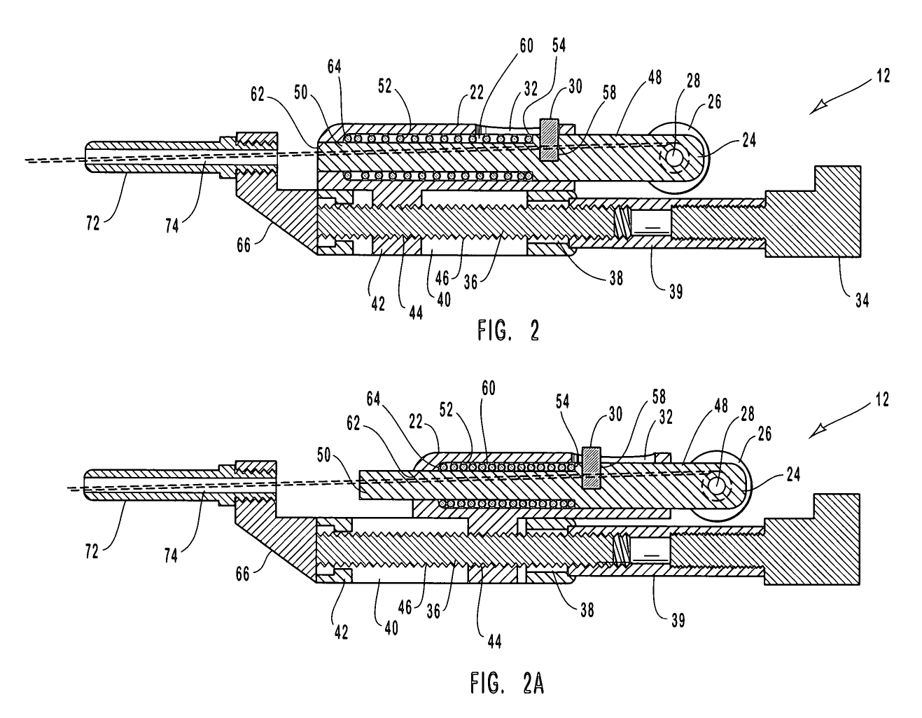

In order to adjust the tension applied by each adjustable tension applicator 18 or 20, a mechanism for selectively moving the cylinder module 22 towards or away from the tensioning piston 22 is provided. As seen in FIGS. 2 and 2A, each adjustable tension applicator 18 or 20 includes a tension adjustment knob 34 attached to a tension adjustment bolt 36 in threaded communication with the cylinder module 22. The tension adjustment bolt 36 passes through a pair of bolt holes 38 at the front and back threaded and thus allow free rotation of the tension adjustment bolt 36 without changing the location of the tension adjustment bolt 36 relative to the tensioning module 16. For ease of use, and to conveniently extend the tension adjustment knobs 34 behind or beyond the tensioning pistons 24, knob extenders 39 may be provided as shown in both FIGS. 1 and 2.

Beyond each of holes 38, each tension adjustment bolt 36 is suspended within a cylinder block guide cavity 40, which holds and guides the cylinder module 22 as it slides back and forth relative to the tensioning block 16 and the tensioning piston 24. More particularly, a side tongue or extension 42 extending laterally from the bottom of the cylinder module 22 is able to slide back and forth within the cylinder block guide cavity 40. The side extension 42 of the cylinder module 22 further includes a threaded hole 44 in threaded communication with the tension adjustment bolt 36, which includes corresponding threads 46. The interaction between the adjustment bolt threads 46 and the threaded hole 44 of the cylinder module 22 provides for fine, adjustable movement of the cylinder module 22 relative to the tensioning piston 24 as the tension adjustment bolt 36 is selectively rotated, such as by means of the tension adjustment knob 34. The degree or magnitude of movement of the cylinder module 22 per revolution of the tension adjustment bolt 36 is dependent on the gauge of the threads 44 and 46.

As seen in FIGS. 2 and 2A, the tensioning piston 24 further includes a first piston end 48 having a first diameter and a second piston end 50 having a second diameter that is smaller than the diameter of the first piston end 48. A biasing spring 52 is circumferentially disposed around the second piston end 50 and makes abutment with an internal end face 54 of the first piston end 48. As better seen in FIG. 1, the tensioning piston 24 also includes a longitudinal guide pin hole 56 through which a guide pin can pass, if desired, during attachment of the tensioning device 10 to the patient's limb. The tensioning piston 24 also includes an attachment hole 58 into which the tension indicator pole 30, is mounted.

The cylinder module 22 includes an internal cylindrical hollow 60 having a diameter that is complementary to the diameter of the first piston end 48 so as to allow for slidable passage of the first piston end 48 therethrough as the cylinder module 22 is moved either towards or away from the tensioning piston 24. The cylinder module 22 further includes a smaller diameter end hole 62 sized so as to allow for slidable passage of the smaller diameter second piston end 50 therethrough as the cylinder module 22 is moved either towards or away from the tensioning piston 24. The biasing spring 52 that is circumferentially disposed around the smaller diameter second piston end 50 of the tensioning piston 24 makes abutment with an internal end face 64 of the internal cylindrical hollow 60 at the junction with the end hole 62.

Thus, the biasing spring 52 is maintained within the length or volume defined by the internal end face 64 of the internal cylindrical hollow 60 and the internal end face 54 of the fist piston end 48 of the tensioning piston 24. In this way, the biasing spring 52 becomes compressed as the cylinder module 22 is moved towards the tensioning piston 24 (as seen in FIG. 2A), thereby increasing the compressing force applied by the biasing spring 52 onto the tensioning piston 24, which is essentially equal to the tensile load applied by the tension piston 24 onto the soft tissue graft attached thereto.

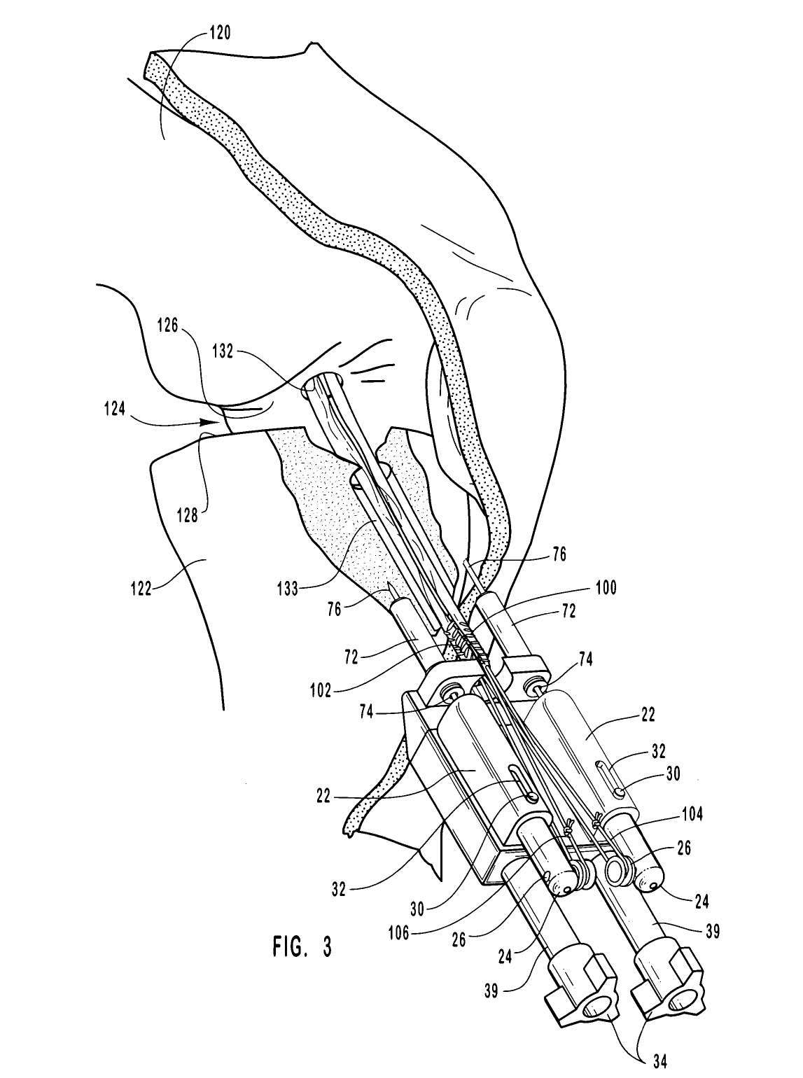

The tensioning system 12 is advantageously attached to the patient's limb (e.g., the leg below the knee) by means of the limb attachment system 14. As seen in FIGS. 1, 2 and 2A, the limb attachment system 14 includes a limb attachment block or module that is matable with the tensioning block or module 16. The limb attachment module 66 further includes a pair of pin guides 72, each having a longitudinal guide pin hole 74 therethrough sized so as to accommodate a guide pin 76 (FIG. 3). The guide pins 76 can be driven, drilled or otherwise pushed into the bone of the patient's limb.

Once the guide pins 76 have been attached to the bone, the limb attachment module 66 can be conveniently slid on and off the guide pins 76 as desired. Once the attachment module 66 has been attached to the patient's limb, the tensioning system 12 can be attached to the limb attachment system 14. Even though the limb attachment module 66 is only slidably connected to the guide pins 76, the tensioning device 10 is held in place against the patient's limb by the countervailing tension exerted by the soft tissue graft being tensioned.

In an exemplary method for carrying out joint repair procedure, two or more strands comprising a soft tissue graft are harvested from the patient, such as from the ham strings or patellar tendon. In one embodiment, the semitendinous and gracillis are harvested from the patient's body. As shown in FIG. 3, the soft tissue graft may comprise a first soft tissue strand 100 and a second soft tissue strand 102. The ends of the soft tissue strands 100 and 102 opposite the free ends (or the ends where tension is to be applied) are attached at an appropriate location on the patient's bone comprising one of the bones of the joint. A looped tissue graft will include a pair of free ends. First graft attachment sutures 104 are attached to the free end(s) of the first soft tissue strand 100 and second graft attachment sutures 106 are attached to free end(s) of the second soft tissue strand 102. Each attachment suture 104 and 106 includes a pair of free ends tied together so as to form a loop, which is in turn looped around a corresponding suture attachment wheel 26. The suture attachment wheels 26, if allowed to freely rotate, equalize the tension applied to each side of looped sutures 104 and 106.

Each tension adjustment knob 34 is independently operated as desired to apply a desired tensile load to each of first and second soft tissue strands 100 and 102. The magnitude of the tensile load being applied to each soft tissue strand 100 and 102 can be measured by the displacement of each tension indicator pole 30 relative to its respective tension indicator slot 32, e.g., by referencing the location of each tension indicator pole 30 in relation to corresponding graduations 33 on the side of the corresponding tension indicator slot 32.

After the soft tissue strands 100 and 102 of the soft tissue graft have been properly conditioned and pre-tensioned, they are advantageously anchored or otherwise attached to the tibia 122. Anchoring may be accomplished, for example, by means of an interference screw (not shown). After securing the soft tissue strands 100 and 102 of the soft tissue graft to the tibia 122, the tensioning device 10 is removed by cutting or otherwise separating the sutures 104 and 106 from the suture attachment wheels 26 and then sliding the tensioning device 10 off of the guide pins 76. Thereafter, the guide pins 76 are removed from the patient's tibia by known surgical procedures.

FIGS. 4-6 illustrate an exemplary suture pulley assembly 150 within the scope of the invention. FIG. 4 is an exploded view of the suture pulley assembly 150, showing its constituent parts. FIGS. 5 and 6 depict the suture pulley assembly 150 in assembled form. Suture pulley assembly 150 includes an outer pulley plate 152 and an inner pulley plate 154 that comprise a pulley wheel 26 and that define a pulley space therebetween into which a looped suture strand 168 can be placed (see FIG. 5) so as to attach the looped suture strand 168 to an adjustable tension applicator of a graft tensioning device.

As best seen in FIGS. 4 and 6, the pulley wheel 26 is rotatably attached to an end of an adjustable tension applicator 156 by means of a post 158. The post 158 can be fixedly or removably attached to the adjustable tension applicator 156. The post 158 passes through a central recess 160 of each pulley plate. The post 158 further includes a flange 159 that overlaps at least a portion of an outer surface of the outer pulley plate 152. In this way the flange 159 prevents the pulley wheel 26 from inadvertently slipping off from the post 158. A sleeve 162 is disposed around at least a portion of the post 158 so as to lie between an outer surface of post 158 and an inner circumferential edge of each pulley plate defining the central recess 160. A spring 164 is disposed around a portion of the sleeve 162 and is sandwiched between a washer 166 adjacent to the end of the adjustable tension applicator 156 and inner pulley plate 154. Spring 164 is therefore positioned so as to bias the inner pulley plate 154 away from the washer 166 and toward outer pulley plate 152. In this way the inner pulley plate 154 and outer pulley plate 152 are oppositely biased (or spring biased).

Whereas the outer pulley plate 152 is not itself spring biased, a force opposite the force of spring 164 is exerted onto the outer pulley plate 152 by the flange 159. In this way, inner pulley plate 154 and outer pulley plate 152 are oppositely biased. A second spring (not shown) may optionally be provided in order to actively bias outer pulley 152 toward inner pulley 154.

As best shown in FIG. 5, the pulley wheel 26 is able to rotate so as to equalize the tensile load or force applied to each end of a looped suture strand 168. In order to assist the practitioner in inserting the looped suture strand 168 into the pulley space defined by the pulley plates 152 and 154, outer pulley plate 152 and inner pulley plate 154 each include an inner surface 170 that is angled so that at least a portion of the pulley space has increasing width toward the outer perimeter of the pulley plates 152 and 154. This creates a V-shaped channel that feeds the looped suture strand 168 toward an interior pulley space 172 having a constant width. A stop 174 may be positioned between pulley plates 152 and 154 in order to maintain. a desired space between the pulley plates 152 and 154. As long as the looped suture 168 has a diameter that is no larger than stop 174, the pulley plates 152 and 154 will remain in a spaced-apart orientation defined by the width of stop 174. However, when a looped suture strand 168 having a larger diameter, such as where a knot (not shown) is included, inner pulley plate 154 is able to retract away from outer pulley plate 152, thereby increasing the width of the pulley space, in order to accommodate the increased width or diameter of the suture strand. This helps maintain good seating and equalization of force on the two sides of the looped suture strand 168.

According to another aspect of the suture pulley assembly 150, the inner pulley plate 154 and outer pulley plate 152, being oppositely biased, are able to expand and then contract depending on the spreading force of the suture strand and any suture knots, as well as any half knots that are formed intermediate of the finished suture knots. In this way, the inner pulley plate 154 and outer pulley plate 152 are able to expand and then contract around any half knots (not shown) that may be formed during formation of the suture knots, thereby holding the half knot white the remaining portion of the suture knot is being tied. This obviates the need for external holding devices (e.g., suture clamps) to hold the half knot while the remaining portion of the suture knot is being tied.

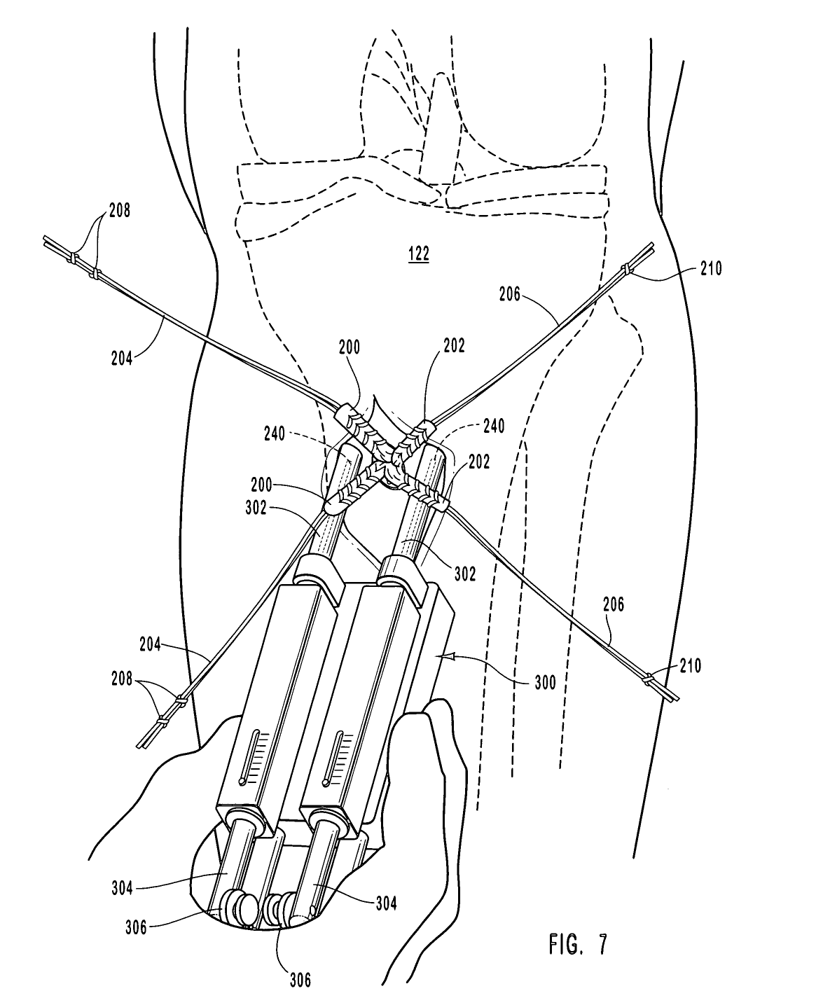

FIG. 7 illustrates a non-modular graft tensioning device 300 positioned adjacent to a bone tunnel and attached to a pair of soft tissue graft strands 200 and 202. The tensioning device 300 includes a pair of hollow attachment posts 302 that are slidably inserted over a pair of guide pins 240 extending from the tibia 122. In this way the tensioning device 300 can be slidably connected to the guide pins 240. The free ends of the soft tissue graft strands 200 and 202 extend out from the tibial bone tunnel to corresponding suture strands 204, 206. Knots 208, 210 help identify to which of the grafts 200 or 202 the respective suture strands 204, 206 are attached.

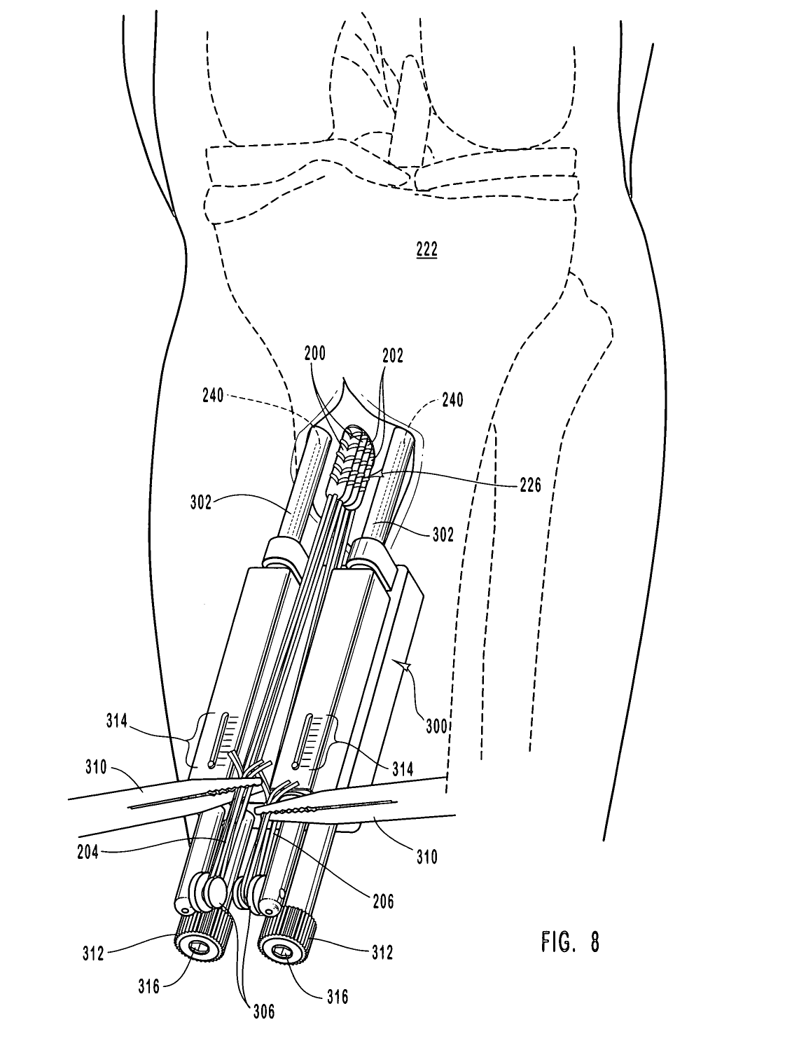

FIG. 8 depicts a method in which the suture strands 204, 206 are each clamped after being looped around a respective suture pulley wheel 306 by means of a respective suture clamp 310. If the suture clamps 310 are the only means of joining the free ends of the suture strands 204, 206 together, they may be left in place throughout the entire procedure until the free ends of the tissue graft strands 200, 202 have been secured to the tibia 122, e.g., within the tibial bone tunnel. The initially free ends of suture strands 204 and 206 may alternatively be tied together with suture knots to form looped suture strands 204 and 206.

The suture pulley wheel 306 is advantageously oppositely-biased (e.g., spring biased with respect to one or both of inner pulley plate 152 and outer pulley plate 154) in order to accommodate varying widths within the suture strands 204 and 206, particularly with respect to accommodating the size and/or assisting in the tying of knots 208 and 210 and/or other knots that might be tied (e.g., to form looped suture strands). In one embodiment, the suture pulley wheel 306 comprises suture pulley assembly 150, as depicted in FIGS. 4-6 and described above.

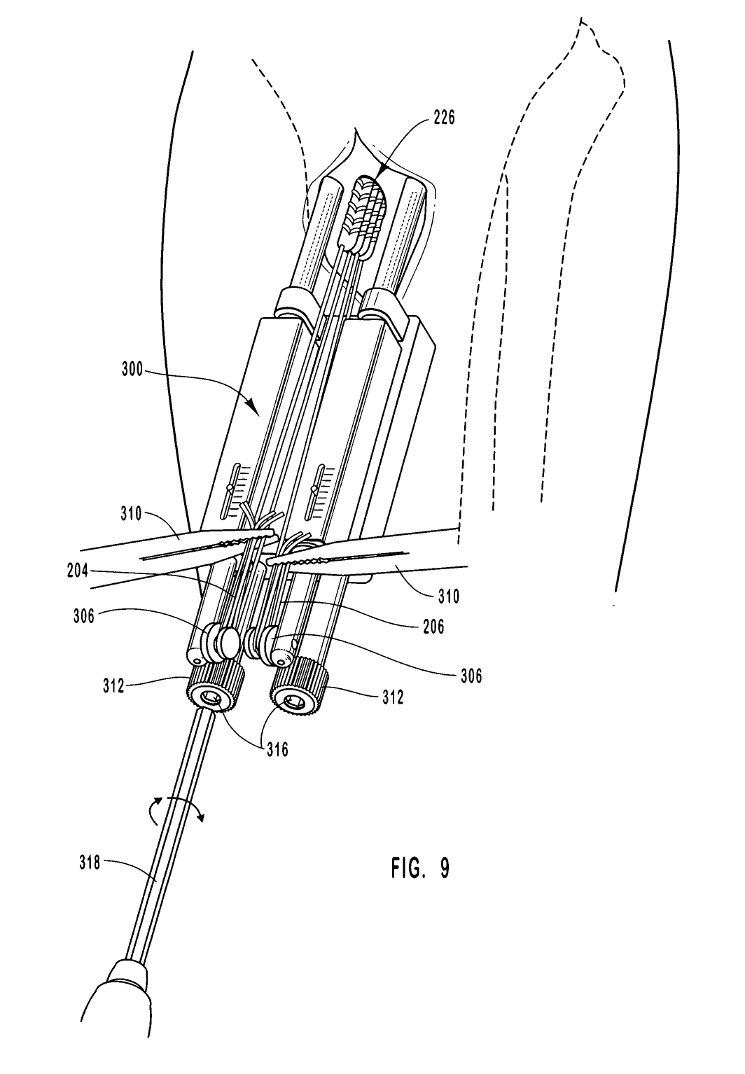

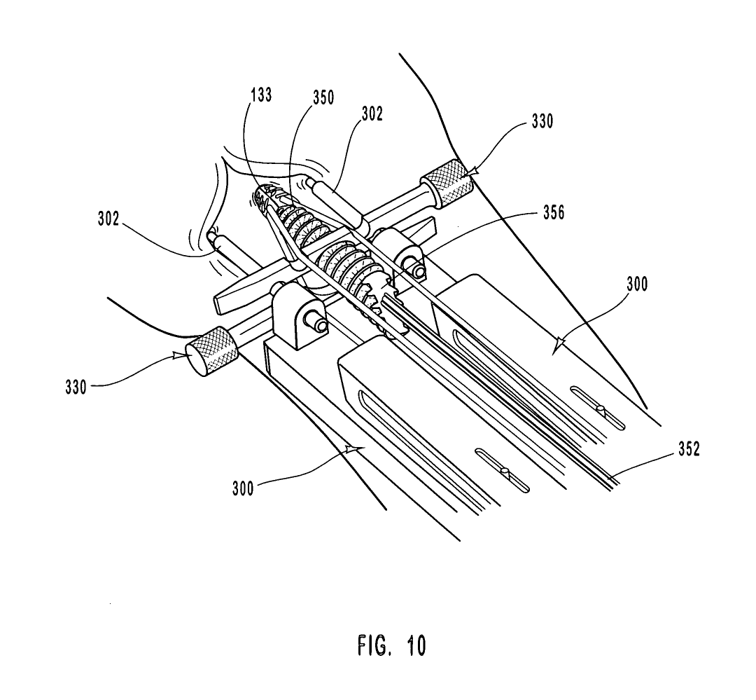

The tensioning device 300 includes a pair of tension adjustment knobs 312, which interact with corresponding tensioning pistons 304 attached to the suture pulley wheels 306. By rotating the tension adjustment knobs 312, as illustrated in FIG. 9, an individualized tension or tensile stress can be separately applied to each soft tissue graft strand 200 and 202. The tension that is individually applied to each graft strand 200, 202 can be determined by viewing a tension gauge 314 associated with each tensioning piston 304. The tension adjustment knobs 312 may optionally include a hexagonal shaped recess 316 or other appropriate recess, protrusion, or other mechanical feature that permits attachment of a driver 318 to the tension adjustment knob 312. This assists the user in applying a desired level of tension.

According to one embodiment, as illustrated in FIG. 11, the suture strands 204, 206 are separated into four quadrants by means of a pair of suture strand separators 330. This, in turn, provides more clear access to the tibial tunnel 133 for insertion of an interference screw 350 or other anchoring device. An interference screw 350 attached to an appropriate driver 352 is inserted through the central recess 340 of the suture strand separators 330 and screwed into the tibial bone tunnel 133. The interference screw 350 advantageously includes a recess designed to receive therein a correspondingly-shaped driving end of the driver 352. In one embodiment, the interference screw 350 may include an angled face 356 designed so as to lie substantially flush with the tibia when screwed into the tibial tunnel 133. This obviates the need to cut or remove part of the interference screw 350. It is, of course, within the scope of the invention to remove (e.g., by cutting) any excess portion of the interference screw 350 that extends beyond the tibia 122. Once the interference screw 350 or other securing means has been used to secure the tissue graft 226 to the tibia 122, the tensioning device 300 may be removed. The guide pins 240 are then removed and properly disposed of.

The present invention may be embodied in other specific forms without departing from its spirit or essential characteristics. The described embodiments are to be considered in all respects only as illustrative and not restrictive. The scope of the invention is, therefore, indicated by the appended claims rather than by the foregoing description. All changes which come within the meaning and range of equivalency of the claims are to be embraced within their scope.

1. In a graft tensioning device for use in joint repair surgery, a suture pulley assembly comprising:
a pulley wheel comprising first and second pulley plates sized and positioned so as to define a pulley space therebetween;
attaching means for rotatably attaching said pulley wheel to an adjustable tension applicator of the graft tensioning device; and
biasing means for biasing at least one of said pulley plates toward the other of said pulley plates.

2. A suture pulley assembly as defined in claim 1, said attachment means comprising a post that passes through a central recess of each pulley plate and that is attached at a first end to the adjustable tension applicator of the graft tensioning device.

3. A suture pulley assembly as defined in claim 2, said post being fixedly attached to the adjustable tension applicator.

4. A suture pulley assembly as defined in claim 2, said post further comprising a flange at a second end opposite said first end and adjacent to one of said pulley plates, said flange overlapping at least a portion of an outer surface of said pulley plate adjacent to said flange.

5. A suture pulley assembly as defined in claim 1, said biasing means comprising a spring.

6. A suture pulley assembly as defined in claim 5, said spring being positioned between one of said pulley plates and the adjustable tension applicator of the graft tensioning device.

7. A suture pulley assembly as defined in claim 1, said first and second pulley plates spreading apart relative to each other so as to selectively define a larger pulley space in response to tying a half knot within one or more suture strands and then clamp the half knot while a remaining portion of the suture knot is tied.

8. A suture pulley assembly as defined in claim 1, said first and second pulley plates spreading apart relative to each other so as to selectively define a larger pulley space in response inserting a suture having a knot into said pulley space.

9. A graft tensioning device for use in joint repair surgery, comprising:
a suture pulley assembly as defined in claim 1; and
at least one adjustable tension applicator to which said suture pulley assembly is rotatably attached and which is configured to apply a desired tensile load to a looped suture attached to free ends of a looped tissue graft,
said suture pulley assembly equalizing a tensile load applied by said adjustable tension applicator to each side of the looped suture.

10. A graft tensioning device as defined in claim 9, said graft tensioning device comprising two independently adjustable tension applicators and a separate suture pulley assembly as defined in claim 1 rotatably attached to each of said two independently adjustable tension applicators.

11. In a graft tensioning device for use in joint repair surgery, a suture pulley assembly comprising:
a pulley wheel comprising first and second pulley plates sized and positioned so as to define a pulley space therebetween;
a post attached at a first end to an adjustable tension applicator of the graft tensioning device,
said post passing through a central recess of each of said first and second pulley plates so as to rotatably attach said pulley wheel to the adjustable tension applicator; and
a spring positioned relative to at least one of said first and second pulley plates so as to bias at least one of said pulley plates toward the other of said pulley plates.

12. A suture pulley assembly as defined in claim 11, said first and second pulley plates having inner surfaces that define said pulley space.

13. A suture pulley assembly as defined in claim 12, at least a portion of said inner surfaces of said first and second pulley plates being angled so that at least a portion of said pulley space has decreasing width from an outer perimeter of said pulley plates toward a center of said pulley wheel.

14. A suture pulley assembly as defined in claim 13, a portion of said pulley space nearest said center of said pulley wheel having a constant width.

15. A suture pulley assembly as defined in claim 11, said first and second pulley plates spreading apart so as to temporarily define a larger pulley space in response to inserting a suture having a knot or half knot into said pulley space.

16. A suture pulley assembly as defined in claim 11, said post being fixedly attached to the adjustable tension applicator.

17. A suture pulley assembly as defined in claim 11, said post further comprising a flange at a second end opposite said first end and adjacent to one of said pulley plates, said flange overlapping at least a portion of an outer surface of said pulley plate adjacent to said flange.

18. A suture pulley assembly as defined in claim 17, said spring being positioned between one of said pulley plates and the adjustable tension applicator of the graft tensioning device.

19. A suture pulley assembly as defined in claim 17, said spring being positioned between one of said pulley plates and said flange of said post.

20. A suture pulley assembly as defined in claim 11, further comprising a sleeve disposed around at least a portion of said post between said post and an inner edge of each pulley plate defining said central recess.

21. A suture pulley assembly as defined in claim 20, said spring being disposed around a portion of said sleeve.

22. A suture pulley assembly as defined in claim 11, further comprising a washer disposed between said spring and the adjustable tension applicator of the graft tensioning device.

23. A graft tensioning device for use in joint repair surgery, comprising:
at least one adjustable tension applicator configured to apply a desired tensile load to a looped suture attached to free ends of a looped tissue graft: and
a suture pulley assembly rotatably attached to said adjustable tension applicator so as to equalize a tensile load applied by said adjustable tension applicator to each side of the looped suture, said suture pulley assembly comprising:
a pulley wheel comprising first and second pulley plates sized and positioned so as to define a pulley space therebetween;
a post attached at a first end to an adjustable tension applicator of the graft tensioning device,
said post passing through a central recess of each of said first and second pulley plates so as to rotatably attach said pulley wheel to the adjustable tension applicator; and
a spring positioned relative to at least one of said first and second pulley plates so as to bias at least one of said pulley plates toward the other of said pulley plates.
